(19) United States
(12) Patent Application Publication
(10) Pub. No.: US 2002/0049383 A1
(43) Pub. Date: Apr. 25, 2002

(54) Catheter based arrays for intracardiac ultrasound imaging

(76) Inventors: John W. Swanson, Portland, OR (US); Richard G. Sass, Portland, OR (US); Jerome J. Boogaard, Forest Grove, OR (US); David J. Sahn, Oswego, OR (US); Perry W. Kaminski, Stehekin, WA (US)

Correspondence Address: Timothy E. Siegel, Patent Attorney, Suite 206, 1868 Knapps Alley, West Linn, OR, 97068-4644, US

(21) Appl. No.: 10/023,097

(22) Filed: Dec. 17, 2001

Related U.S. Application Data
(63) Continuation-in-part of application No. 09/459,826, filed on Dec. 13, 1999.
(60) Provisional application No. 60/112,314, filed on Dec. 14, 1998.

(51) Int. Cl. A61B 8/02

(52) U.S. Cl. 600/450

(57) ABSTRACT

An ultrasound probe assembly adapted for bio-imaging. The assembly comprises a catheter, an electrical assembly, including a set of linear conductors, at least partially housed within said catheter. An ultrasound transceiving unit is electrically connected to said electrical assembly and can be placed in both an undeployed state in which the transceiving unit is oriented coincidentally to said catheter and a deployed state in which it is directed to provide imaging signals over a volume of interest.

### BACKGROUND OF THE INVENTION

The present invention is an apparatus and method for introducing an ultrasound transceiver into a body cavity such as the right atrium of the heart in order to image a portion of the body, such as the left ventricle of the heart.

The mammalian heart typically has four chambers: Two ventricles for pumping the blood and two atria, each for collecting the blood from the vein leading to it and delivering that blood to the corresponding ventricle when it is not pumping. The left ventricle pumps blood to the vast bulk of the mammalian body. As a result, problems with the left ventricle or with the mitral valve, which leads from the left atrium into the left ventricle, can cause serious health problems for an affected individual. When it appears that a patient has inadequate blood circulation in a portion of his or her body, the left ventricle and the mitral valve are naturally suspect. Specifically diagnosing the problem to these structures, however, and more specifically to a particular problem with the left ventricle or mitral valve is not an easy proposition. In fact unnecessary surgeries are sometimes performed due to the difficulty of forming a certain diagnosis.

More generally, heart disease, including ischemic, valvular and arrhythmias, represents one of the most common debilitating disease complexes in the elderly of the US, and is a common cause of death. A number of therapies have been created for treating cardiac conditions by way of the introduction of a laser or other device into a chamber of the heart by way of a catheter. One of the most active areas, in need of the most sophisticated approach to ultrasound intracardiac imaging, involves the transcatheter attempts to produce linear and continuous electrophysiologic lesions on the atrial wall for treatment of chronic atrial fibrillation in elderly adults. 2.2 million people in the U.S.A. have this condition, and it is increasing in frequency as our population ages. In the Maze procedure, a grid of linear interruptions is produced on the atrial wall to treat this condition—most commonly in the right atrium and less commonly the left atrium. Unfortunately, the limitations of currently available cardiac imaging techniques result in these therapies being performed with a great shortage of desirable information.

Accordingly, there are many important purposes for creating images of the heart, including in particular the left ventricle and the mitral valve. Among these purposes is the assessment of the need for corrective cardiac surgery and the informing and guiding of cardiac therapeutic procedures by way of imaging performed during such procedures. Unfortunately, currently available methods for imaging the heart and specifically the left ventricle and the mitral valve leave much to be desired. One method, termed transthoracic imaging, typically requires the placement of an ultrasound transceiver against the chest of the patient and the use of this transceiver to image the heart. Unfortunately, the bones and the other tissue types that are interposed between the ultrasound transceiver and the heart during this procedure prevent the formation of a sufficiently detailed image of the heart. Another cardiac imaging method, trans-esophageal imaging, involves the insertion of an ultrasound transceiver into the esophagus of the patient. Although trans-esophageal imaging places the ultrasound transceiver closer to the heart, the patient must be rendered unconscious by way of a general anesthetic for this method to be employed. As a result, the patient's cooperation is surrendered. In cardiac imaging it is very valuable to have a cooperative patient who can change position upon request in order to facilitate the imaging of different views of the heart under various operating conditions.

Other body cavities could host the distal end of an ultrasound probe, if it were possible to introduce the probe into the body cavity in a form that could be passed through a relatively narrow passageway leading to the cavity. Unfortunately, currently available ultrasound probes are not constructed to have an undeployed state in which the probe is very narrow and amenable to this method of introduction into a body cavity.

### SUMMARY

In a first separate aspect, the present invention is method for imaging at least a portion of a mammalian heart, having a right atrium and residing in a mammal having veins leading to the right atrium. As a preliminary step, an ultrasound probe assembly is provided. This assembly has a proximal end and a distal end and can be placed into both a deployed state and an undeployed state. In the undeployed state the probe assembly can be inserted into a vein leading to the right atrium and thereby into the right atrium. When placed in the deployed state the probe assembly is positioned into an advantageous imaging orientation. The distal end is capable of transmitting and receiving ultrasound signals, converting the received signals into electrical signals and transmitting the received signals to a receiving apparatus. In the next steps, the assembly is placed into its undeployed state and the distal end of the probe assembly is introduced into the right atrium by way of a vein leading to the right atrium. Next the assembly is placed into its deployed state and used to image at least a portion of the heart.

In a second separate aspect, the present invention is an ultrasound probe assembly adapted for bio-imaging. The assembly comprises a catheter, an electrical assembly, including a set of linear conductors, at least partially housed within said catheter. An ultrasound transceiving unit is electrically connected to said electrical assembly and can be placed in both an undeployed state in which the transceiving unit is oriented coincidentally to said catheter and a deployed state in which it is directed to provide imaging signals over a volume of interest.

The foregoing and other objectives, features and advantages of the invention will be more readily understood upon consideration of the following detailed description of the invention, taken in conjunction with the accompanying drawings.

### BRIEF DESCRIPTION OF THE DRAWINGS

FIG. 1 shows a perspective view of preferred embodiment of the ultrasound probe assembly of the present invention, in the right atrium of a human heart.

FIG. 2a shows perspective view of the ultrasound transceiver of the ultrasound probe of FIG. 1.

FIG. 2b shows a partial side view of ultrasound transceiver of FIG. 2a.

FIG. 3a shows a side view of the distal portion of the ultrasound probe assembly of FIG. 1.

FIG. 3b shows a perspective view of the distal portion of FIG. 3a.

FIG. 4a shows a side view of the distal end of an ultrasound probe assembly that represents an alternative preferred embodiment of the present invention.

FIG. 4b shows a top view of the distal end of the present invention.

FIG. 4c shows a perspective view of the distal end of the present invention.

FIG. 5 is a perspective view of an alternative preferred embodiment of an intracardiac imaging device according to the present invention.

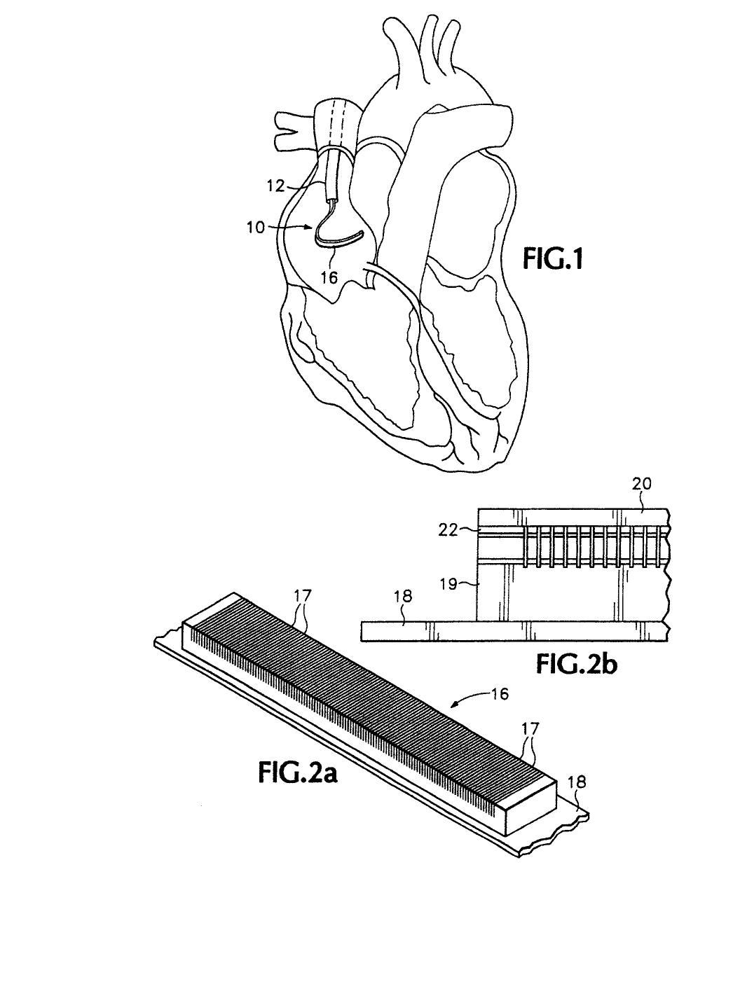

### DETAILED DESCRIPTION OF THE PREFERRED EMBODIMENTS

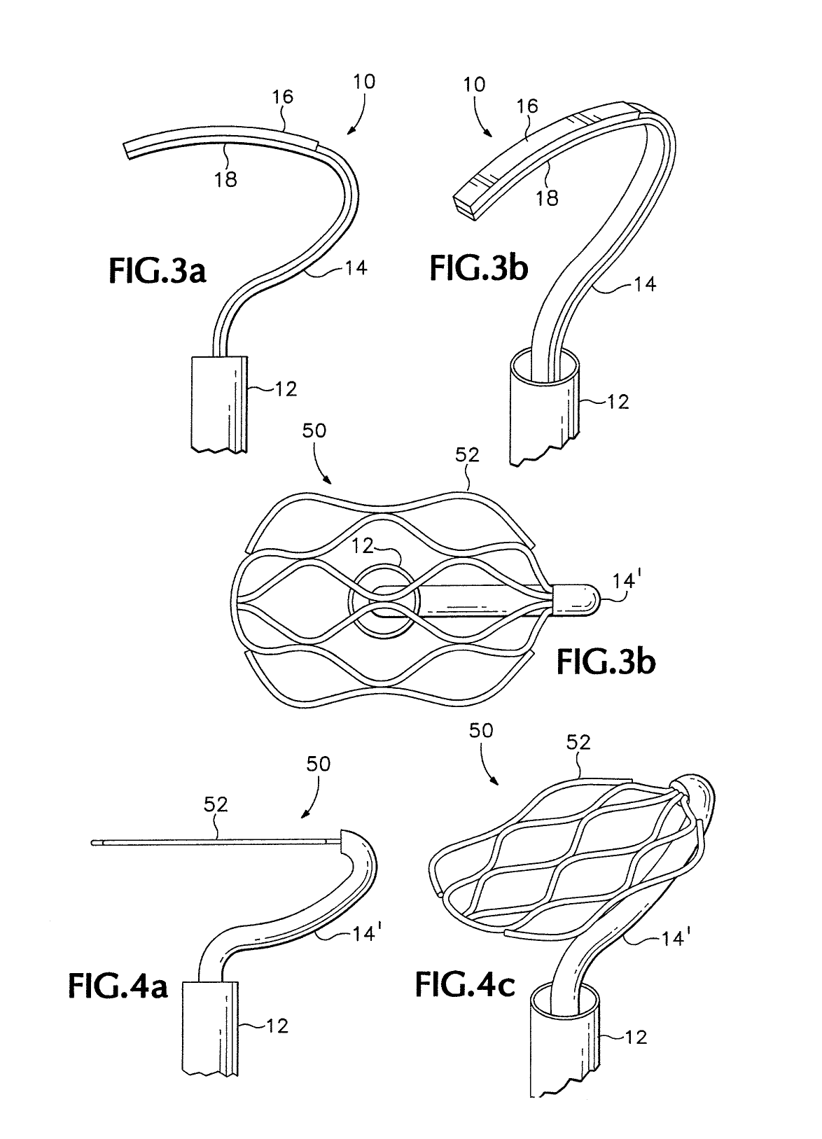

In one preferred embodiment the invention is an ultrasound probe assembly 10 that includes a catheter 12 (in one preferred embodiment a No. 14 French catheter is used) that houses an conductor bearing structure 14 in the form of a stiffened ribbon cable and terminating in an ultrasound transceiver 16 that is a linear array of 128 piezoelectric (preferably ceramic) elements 17 supported by a polymer backing layer 19 that is in turn supported by a substrate of shape memory material 18. An acoustic lens 20, preferably constructed of silicone shapes the ultrasound beam created by elements 17. A polymeric matching layer 22 is interposed between element 17 and the lens 20. In its undeployed state the transceiver 16 is straight, enabling it to fit conveniently within the catheter 12, for introduction into a body cavity, such as the right atrium. To place assembly 10 into its deployed state, however, a human operator pushing on the proximal end of structure 14 pushes the transceiver 16 outwardly from the end of the catheter 12. The warmth of the human body then warms the shape memory material of substrate 18 to the point that it assumes its memorized shape, which is in the form of a hook, as shown. Extending and retracting catheter 12 relative to transceiver 16 steers transceiver 16 in the plane of the paper in FIG. 3a, permitting various views of portions of the heart.

The array of elements 17 may be constructed to operate a center frequency of 9 MHz, with a Doppler range of 6 MHz. The effective aperture length may be 18 mm with an aperture width of 3 mm and a radius of curvature of 20 mm, to produce an effective field of view of 45 to 50 degrees. Lens 20 may have a transverse radius of 10 mm. Elements 17 may be divided into two subdivisions and positioned with a pitch of 0.07 mm.

An alternative preferred embodiment 50 is shown in FIGS. 4a-4c. (In these Figs., structures that are identical with structures already introduced are labeled with the previously introduced reference numbers and structures that are analogous with previously introduced structures are designated by the previously introduced reference number primed. In this embodiment a collection of shape memory strands 52 fans out into a rigid web when warmed to body temperature. Strands 52 bear a collection of piezoelectric elements that can be coordinated together into a large aperture electrically steerable array, thereby collecting data sufficient to permit three-dimensional imaging.

In yet another embodiment a set of strands form the volumetric outline of a bulb when the memorized shape is assumed.

Assembly 10, or assembly 50, is introduced into the right atrium by placing assembly 10 into its undeployed state and introducing it into the jugular vein at the patient's neck. The patient may remain conscious during this procedure, with a local anesthetic being applied to permit the device insertion. The distal end of the assembly is then introduced into the right atrium, where it assumes its deployed state and is used for imaging. The total in vivo travel distance of the assembly 10 is on the order of six to nine inches. Because the patient remains conscious during the procedure, he or she may aid in the imaging process by shifting position during the imaging procedure. To remove, ultrasound transceiver 16 is pulled back into catheter 12, which constrains transceiver 16 to the general shape of catheter 12, permitting the removal of assembly 10 by a simple pulling motion.

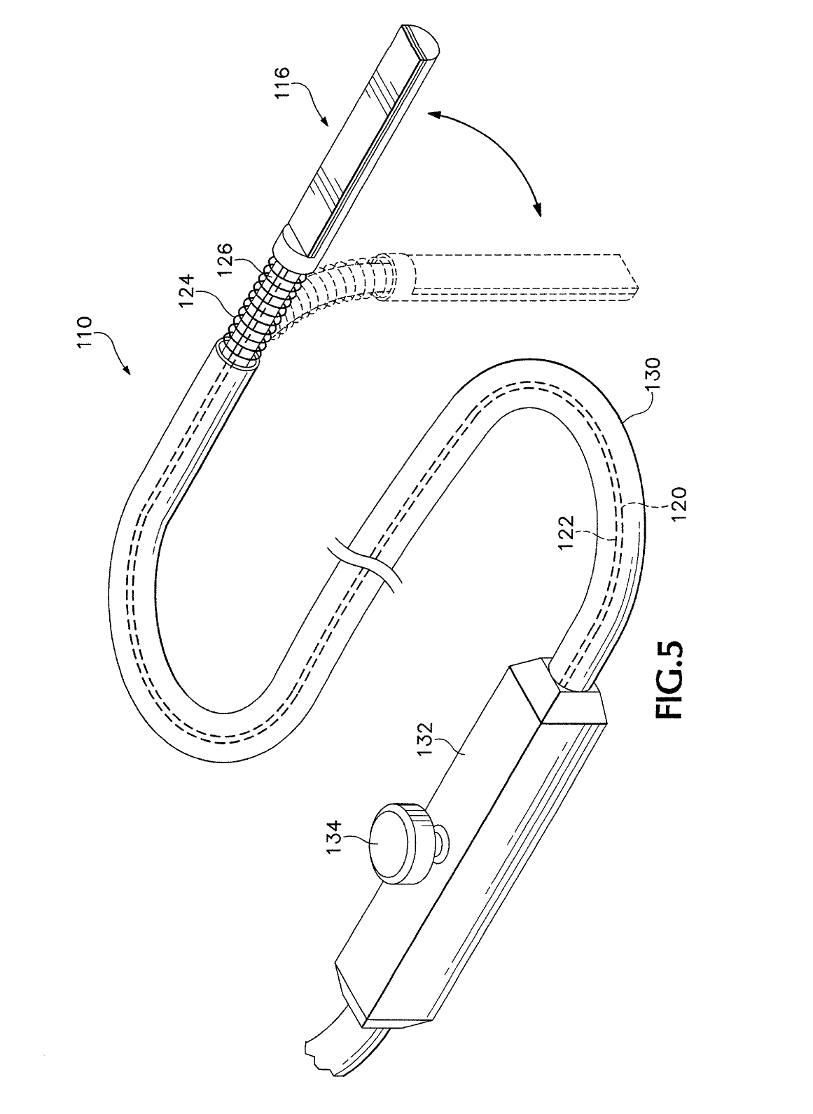

Referring to FIG. 5, in an additional preferred embodiment, an imaging probe 110, includes a piezoelectric array 116, which may be very similar to array 16 (FIG. 2b). Assembly 110, however, is placed into a deployed state (as shown by dashed line portion of figure), by pulling on one or the other of a pair of tension lines 120 and 122. Line 120 pulls array 116 downwardly, whereas line 122 pulls array 116 upwardly in the plane of FIG. 5. A spring 124 urges array 116 into a position that is straight with respect to catheter 130. Catheter 130 includes one hundred and twenty-eight coaxial cables (not shown), one for each piezoelectric element. These coaxial cables are typically bound together inside the catheter 130. At the interior of spring 124, however, the signal paths take the form of traces in a flex circuit 126, which may be flexed up or down as shown in FIG. 5. A fixture 132 and a knob 134, attached to tension lines 122 and 124, is provided to aid a doctor in manipulating array 116.

The terms and expressions which have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims which follow.

1. A method for imaging a portion of a mammalian heart, having a right atrium, said mammalian heart residing in a mammal having veins leading to said atrium, by performing the steps of:
(a) providing an ultrasound probe assembly having a proximal end and a distal end and which can be placed into both a deployed state and an undeployed state and which, in said undeployed state, can be inserted into a said vein leading to said right atrium and thereby into said right atrium and which in said deployed state said distal end is oriented into a more advantageous imaging orientation said distal end being capable of transmitting and receiving ultrasound signals, converting said received signals into electrical signals and transmitting said received signals to a receiving apparatus;
(b) placing said ultrasound probe assembly into its undeployed state;
(c) introducing said distal end of said ultrasound probe assembly into said right atrium by way of a said vein leading to said right atrium;
(d) deploying said ultrasound probe assembly into an advantageous imaging orientation; and
(e) using said deployed ultrasound probe assembly to create electrical signals that may be used to image a portion of said mammalian heart.

2. The method of claim 1 in which said portion of said mammalian heart includes the mitral valve.

3. The method of claim 1 in which said ultrasound probe assembly includes wires for transmitting said electrical signals from said distal end to said proximal end.

4. The method of claim 1 in which said ultrasound assembly includes a flexible spring loaded element near its distal end and is deployed by pulling on a tension element that pulls said distal end of said ultrasound probe assembly, thereby bending said flexible spring loaded element.

5. An ultrasound probe assembly adapted for bio-imaging, comprising:
(a) a catheter;
(b) an electrical assembly including a set of linear conductors, at least partially housed within said catheter;
(c) an ultrasound transceiving unit electrically connected to said electrical assembly and having an undeployed state, in which it is substantially oriented in a first direction, coincidental to said catheter orientation, and having a deployed state in which it is placed into an orientation different from its orientation in said undeployed state and adapted to provide imaging.

6. The ultrasound probe assembly of claim 5 in which said ultrasound transceiving unit includes a substrate of shape memory material.

7. The ultrasound probe assembly of claim 6 in which said shape memory material is nitinol.

8. The ultrasound probe assembly of claim 5 in which said distal end is spring loaded about a flexible element and can be steered by a pair of tension elements.
